SEQUENCE LISTING (8 sequences)

SEQ ID NO: 1 - DNA, 2310 bp, Homo sapiens
  Coding sequence of human APP770 with mutation T714I
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgatagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaac

SEQ ID NO: 2 - DNA, 4958 bp, Artificial Sequence
  construct with APP714
gcgcgcgtaatacgactcactatagggcgaattgggtaccgggccccccctcgaggtcgacggtatcgataagcttgatatcgaattcctgcagcccgggggatccactagaggatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatggccccatctccaggttgaggctagatggaggggacagagggaagtgacttgcccaaggtgacccaaactcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatgaactgaagggttgctcggctctcagagaccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctctctagttctagagcggccagcttggtaccgagctcggatcccccgggctgcaggaattcccgccagggtcgtcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggccacgatgctgcccggtttggcactgctcctgctggccgcctggacggctcgggcgctggaggtacccaccgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgatagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagccctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagctggccgccaccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggattctctagagtcgaccgcggtggagctccagcttttgttccctttagtgagggttaattgcgcgctggcgaat

SEQ ID NO: 3 - DNA, 21 bp, Homo sapiens
gtcatagcgatagtgatcgtc

SEQ ID NO: 4 - DNA, 34 bp, Artificial Sequence
  Primer
cggtgttgtcatagcgatagtgatcgtcatcacc

SEQ ID NO: 5 - DNA, 34 bp, artificial sequence
  Primer
ggtgatgacgatcactatcgctatgacaacaccg

SEQ ID NO: 6 - protein, 5 aa, Artificial Sequence
  synthetic peptide corresponding to amyloid beta peptide residues 36-40
VGGVV

SEQ ID NO: 7 - protein, 10 aa, artificial sequence
  synthetic peptide corresponding to amyloid beta peptide residues 33-42
GLMVGGVVIA

SEQ ID NO: 8 - protein, 7 aa, artificial sequence
  synthetic peptide corresponding to amyloid beta peptide residues 1-7
DAEFRHD

(19) United States
(12) Patent Application Publication
(10) Pub. No.: US 2003/0148356 A1
(43) Pub. Date: Aug. 7, 2003

(54) Novel APP mutation associated with an unusual Alzheimer's disease pathology

(76) Inventors: Mare Cruts, Antwerpen (BE); Chris De Jonghe, Edegem (BE); Samir Kumar Singh, Edegem (BE); Christine van Broeckhoven, Edegem (BE)

Correspondence Address: TRASK BRITT, P.O. BOX 2550, SALT LAKE CITY, UT, 84110, US

(21) Appl. No.: 10/337,970

(22) Filed: Jan. 6, 2003

Related U.S. Application Data
(63) Continuation of application No. PCT/EP01/07830, filed on Jul. 6, 2001.

(51) Int. Cl. C12Q 1/68; C07H 21/04; C12N 9/64; C12N 1/21; C12P 21/02; C12N 15/74

(52) U.S. Cl. 435/6; 435/69.1; 435/226; 435/252.3; 435/320.1; 536/23.2

(57) ABSTRACT

The invention provides a novel mutation identified in amyloid precursor protein, which leads to a very aggressive form of Alzheimer's disease. The mutation involves the 43^{rd} codon of amyloid β peptide (Aβ) corresponding to the putative γ₄₂-secretase cleavage site. The novel mutation alters both Aβ₄₀ and Aβ₄₂ secretion elevating the Aβ₄₂/Aβ₄₀ ratio by 10-fold in vitro. Furthermore, the main amyloid plaque pathology in brains of these patients is of the diffuse ‘pre-amyloid’ type composed primarily of N-truncated Aβ₄₂. Dense-cored plaques although not absent, were significantly reduced. Also, usual sites in brain where Aβ₄₀ is predominantly deposited, for instance, in vessels such as cerebral amyloid angiopathy or senile plaque cores, were composed entirely of Aβ₄₂ form. Together, these indicate that deposition of N-truncated Aβ₄₂ in one of the earliest amyloid deposited in brain, the diffuse plaques, is fully competent of inciting AD through the well-established ‘amyloid cascade’ or by yet unknown mechanism(s).

### RELATED APPLICATIONS

This application is a continuation of co-pending International Application No. PCT/EP01/07830, filed Jul. 6, 2001, designating the United States of America published in English as WO/02/02769 A1 on Jan. 10, 2001, the contents of which are incorporated by this reference.

### TECHNICAL FIELD

The invention relates to the field of Alzheimer's Disease (AD). In particular, the invention provides a novel mutation (T714I) identified in the amylqid precursor protein (APP), APP714, which leads to a very aggressive form of AD. The mutation involves the 43^{rd} codon of the amyloid β peptide (Aβ) corresponding to the putative γ₄₂-secretase cleavage site. The novel mutation alters both Aβ₄₀ and Aβ₄₂ secretion elevating the Aβ₄₂/Aβ₄₀ ratio by 10-fold in vitro. Furthermore, the main amyloid plaque pathology in brains of these patients is of the diffuse ‘pre-amyloid’ type composed primarily of N-truncated Aβ₄₂. Dense-cored plaques although not absent, were significantly reduced. Also, the usual sites in brain where Aβ₄₀ is predominantly deposited, for instance, in vessels as cerebral amyloid angiopathy (CAA) or senile plaque cores, were composed entirely of Aβ₄₂ form. Together, these indicate that deposition of N-truncated Aβ₄₂ in one of the earliest amyloid deposited in brain, the diffuse plaques, is fully competent of inciting AD either through the well-established ‘amyloid cascade’ or by a yet unknown mechanism(s).

### BACKGROUND

Alzheimer's disease is a progressive neurodegenerative disorder defined histologically by the formation in the brain of intracellular neurofibrillary tangles and extracellular amyloid deposits. Particular attention has been focused on the role that the amyloid β-protein (Aβ) plays in the development of AD. Indeed, the predominant protein component of the cortical and cerebrovascular amyloid deposits of AD is the Aβ. Accumulating evidence suggests that Aβ production from amyloid precursor protein (APP), its aggregation into fibrils and its deposition are key etiological events in AD. An understanding of these critical steps will be crucial in determining therapeutic targets. There are at least five distinct isoforms of APP: 563, 695, 714, 751, and 770 amino acids, respectively (Wirak et al. (1991) Science 253:323, the contents of which are incorporated by reference herein, in their entirety). These isoforms of APP are generated by alternative splicing of primary transcripts of the APP gene, which is located on human chromosome 21.

The APP protein is processed by B secretase (BACE) and as yet unidentified α secretase leading to soluble APP (APPs_{α} and APPs_{β}) and membrane-bound C-terminal fragments (α and β CTFs). While cleavage by β- and one or many γ-secretase(s) (γ₄₀ and γ₄₂) releases 40-42 amino acids Aβ peptides (Aβ₁₋₄₀ and Aβ₁₋₄₂), the major secretory pathway utilizes α secretase that cleaves the Aβ sequence between amino acid 16 and 17 of Aβ. Further processing of the αCTFs by γ₄₀ or γ₄₂ secretases releases N-terminally cleaved Aβ₁₇₋₄₀ or Aβ₁₇₋₄₂ peptides (p³). In addition to Aβ_{17-X}, two other major peptides resulting from activity of β secretase are noted to be secreted by transfected cells and deposited in brain starting from amino acid 5 (Aβ_{5-X}) and 11 (Aβ_{11-X}).

As yet, eight missense mutations were identified in APP in families with autosomal dominant early-onset AD. All these mutations are clustering in close proximity of the secretase cleavage sites and affect APP metabolism in two distinct ways. The K670N/M671L mutation located near the B-secretase cleavage site, increases the production of both Aβ₄₀ and Aβ₄₂. In contrast, mutations located near the γ-secretase cleavage sites result in an increased absolute or relative production of Aβ₄₂ while the total amount of Aβ is either unaffected as in APP V717I or decreased as in APP V715M. In contrast, mutations in the presenilin genes (PSEN1 and 2) causing autosomal dominant early onset AD, lead to a preferential increase in Aβ₄₂. This is of consequence to AD pathology as in vitro Aβ₄₂ was demonstrated to be more fibrillogenic and to aggregate faster than Aβ₄₀.

Immunohistochemistry showed that, although Aβ₄₂ is deposited first as diffuse plaques in AD and in Down's syndrome (DS) patients, Aβ₄₀ contributes to further growth of plaques resulting in the formation of dense-cored senile plaques. Aβ₄₀ is also the predominant constituent of the amyloid deposits in blood vessel walls. The amyloid deposited in congophilic dense-cored plaques is certainly fibrillar as it demonstrates Thioflavin-T and Congo red binding. Since most of the neuritic pathology in brain is in association with dense-cored plaques, such congophilic deposits are considered pathogenic in AD and DS. However, the precise significance of nonfibrillar diffuse plaques, suggested by their non-congophilic nature, to disease pathogenesis and cognitive decline in AD and DS patients is poorly understood. These two plaque forms also differ with respect to their precise Aβ content. In contrast to the compact plaques where full-length Aβ is the chief constituent, the diffuse plaques are composed of p3 or other N-truncated Aβ forms. The benign nature of these N-trucated forms of Aβ is also suggested by absence of domains involved in microglial activation and apolipoprotein E (APOE) binding. In this respect, an isoform of APOE predisposes carriers of this allele to the common late-onset form of the disease.

At present, no known effective therapy exists for Alzheimer's disease. It is important to develop experimental models of AD that can be used to define further the underlying biochemical events involved in AD pathogenesis. Such models can be employed, in one application, to screen for molecules that inhibit, prevent, or reverse the progression of AD.

### DISCLOSURE OF THE INVENTION

The present invention provides a novel, mutated nucleic acid encoding human amyloid precursor protein 770 wherein the nucleic acid at codon 714 encodes an Isoleucine (I) instead of Threonine (T). This mutation (APPT714I) is the most ‘drastic’ mutation in APP described so far. In the brain of AD patients with this mutation, the main amyloid plaque pathology is of non-congophilic pre-amyloid nature composed primarily of N-truncated Aβ₄₂ (Aβ_{X-42}) in the absence of Aβ₄₀. These observations implicate a key role for diffuse pre-amyloid Aβ_{X-42}, rather than, or in addition to, the fibrillar full-length forms of Aβ₄₂ and Aβ₄₀, in neuronal toxicity and cognitive decline in AD patients. Whatever the precise pathogenic mechanisms by which T714I causes AD, it is likely that this unusual APP mutation will help elucidating the mechanism(s) through which AD occurs.

The present invention provides an isolated polynucleotide sequence encoding a codon 714 mutant of human amyoid precursor protein 770, which mutant was identified in patients with a very aggressive form of Alzheimer's disease. In particular, codon 714 of APP770 codes for leucine.

The invention further provides a transgenic eukaryotic primary cell, embryonic stem cell line or an immortalized cell line comprising a codon 714 mutant of APP770. The invention also aims at providing a non-human transgenic animal that expresses the codon 714 mutant of APP. Another aim of the invention is to provide methods for screening, in cells and animals, of molecules that can reduce the formation of (N-truncated) beta-amyloid 42 peptide. The invention further aims at using the non-human transgenic animal for the analysis and/or interference of the formation of ‘cloudy’ diffuse plaques. Finally, the invention aims at providing an assay for the screening of alternative proteases of amyloid beta, gamma-secretase homologues and/or gamma-secretase modulators.

### BRIEF DESCRIPTION OF THE DRAWINGS

FIG. 1: (A) Pedigree of AD156 segregating the APP T714I mutation. Solid symbols indicate affected individuals; †: age at death; arrow denotes the proband where autopsy was performed. (B) Sequence analysis for 156.1 and 156.2 showing a heterozygous C to T transition at position 2208 of the cDNA leading to an amino acid substitution of threonine (T) to isoleucine (I) at codon 714 in exon 17 of APP (numbering according to APP770 isoform) (SEQ ID NO: 3. (C) PCR-RFLP analysis of PCR amplified APP exon 17 product followed by TspRI digestion.

FIG. 2: Analysis of soluble APP (APPs_{α} and APPs_{β}). Supernatants were resolved on a NuPAGE gel, and imnimunoblotted with mAb 6E10 for APPs_{α} (A) or 53/4 for APPs_{β} (B). Bands for T714I and WT were quantified using the NIH Image software package (data not shown).

FIG. 3: (A) Representative MALDI-TOF mass spectra for APP WT and T714I. Conditioned medium of HEK293T cells transfected with T714I and WT APP were studied by IP/MS using mAb 4G8. Relative peak intensities were normalized with synthetic Aβ (12-28) peptide (marked: std) and identities of the observed peaks were inferred as described in Methods. Note that predominant peaks for peptides ending at residue 40 are clearly lost for T714I (B) Effect on secreted Aβ with different N and C termini were analysed in detail. These were peptides ending at Aβ₄₂, Aβ₄₀, Aβ₃₉, Aβ₃₈, and Aβ₃₇ and beginning at E1 (Aβ_{1-X}), R5 (Aβ_{5-X}), E11 (Aβ_{11-X}), and L17 (Aβ_{17-X}). Bars are equally scaled to allow inter-panel comparison. Absence of few bars in the WT is due to peaks too low to be measured. *: statistical significance of at least <0.001 versus WT.

### DETAILED DESCRIPTION OF THE INVENTION

The present invention provides model systems of Alzheimer's disease, wherein the model system comprises a DNA sequence encoding an amyloid precursor protein (APP) isoform or fragment that has an amino acid other than threonine at the amino acid position corresponding to amino acid residue position 714 of APP770.

The APPT714I mutation in an Austrian pedigree is the first APP mutation reported to date that involves amino acid 43 of Aβ located directly at the γ₄₂ secretase cleavage site. Patients carrying this mutation have a very early onset of AD around 34-35 years, the earliest onset age ever reported for autosomal dominant AD associated with mutations in APP. The early onset age, as well as rapid progression of the disease, and early death, is comparable to AD caused by mutations in PSEN1.

In vitro, the T714I mutation drastically affected the γ-secretase cleavage of APP significantly decreasing Aβ₄₀ and increasing Aβ₄₂. This effect was not limited to full-length Aβ, but also involved N-truncated Aβ peptides. Besides a major effect in altering secretion of Aβ_{X-40} and Aβ_{X-42}, T714I mutation also lead to significant alternative processing of APP to Aβ peptides ending at residues G38 (Aβ₃₈) and V39 (Aβ₃₉). The increase by ˜2.5-fold of total Aβ (Aβ_{X-X}) and an unaltered APPs_{α} and APPs_{β} indicates that processing of CTFs can be a rate-limiting step in APP processing.

A number of terms and expressions are used throughout the detailed description and, to facilitate the understanding thereof, the following definitions are provided:

As used herein, the words “an isolated polynucleotide sequence” preferably means the DNA and cDNA sequence as detailed by Yoshikai et al. (1990) Gene 87:257, together with the promoter DNA sequence as described by Salbaum et al. (1988) EMBO J. 7(9): 2807 (the contents of each of which are incorporated by reference herein, in their entirety).

As used herein, “isoform”, “APP”, and “APP isoform” refer to a polypeptide that is encoded by at least one exon of the APP gene (Kitaguchi et al. (1988) Nature 331:530; de Sauvage and Octave (1989) Science 245:651; Golde et al. (1990) Neuron 4:253, the contents of each of which are incorporated by reference herein, in their entirety). An APP isoform may be encoded by an APP allele (or exon thereof) that is associated with a form of AD or that is not associated with an AD disease phenotype.

As used herein, “fragment” refers to a polypeptide of at least about 9 amino acids, typically 50 to 75, or more, wherein the polypeptide contains an amino acid core sequence.

A fragment may be a truncated APP isoform, modified APP isoform (as by amino acid substitutions, deletions, or additions outside of the core sequence), or other variant polypeptide sequence, but is not a naturally-occurring APP isoform that is present in a human individual, whether affected by AD or not. If desired, the fragment may be fused at either terminus to additional amino acids, which may number from 1 to 20, typically 50 to 100, but up to 250 to 500 or more.

As used herein, “APP770” refers to the 770 amino acid residue long polypeptides encoded by the human APP gene.

As used herein, “codon 714” refers to the codon (i.e., the trinucleotide sequence) that encodes the 714th amino acid position in APP770, or the amino acid position in an APP isoform or fragment that corresponds to the 714th position in APP770. For example but not limitation, a 670 residue long fragment that is produced by truncating APP770 by removing the 100 N-terminal amino acids has its 614th amino acid position corresponding to codon 714.

As used herein, “human APP isoform or fragment” refers to an APP isoform or fragment that contains a sequence of at least 9 consecutive amino acids that is identical to a sequence in a human APP770, APP751, or APP695 protein that occurs naturally in a human individual, and wherein an identical sequence is not present in an APP protein that occurs naturally in a non-human species.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked and, where necessary to join two protein coding regions, contiguous and in reading frame.

As used herein, the term “mutant” refers to APP alleles having missense mutations that are pathognomonic for a genetic predisposition for developing AD; specifically a mutation at codon 714 (as referenced by the amino acid sequence in APP770) of the APP gene, such that codon 714 encodes one of the nineteen amino acids that are not threonine (i.e., valine, glycine, methionine, alanine, serine, isoleucine, leucine, proline, histidine, cysteine, tyrosine, phenylalanine, glutamic acid, tryptophan, arginine, aspartic acid, asparagine, lysine, and glutamine), but preferably isoleucine. Thus the mutant APP770 polypeptide of the present invention is an APP770 polypeptide that has an amino acid residue at position 714 that is not threonine. Other mutant APP isoforms comprise a non-threonine amino acid at the amino acid residue position that corresponds to codon 714 (i.e., that is encoded by codon 714). Similarly, a mutant APP allele or a variant APP codon 714 allele is an APP allele that encodes an amino acid other than threonine at codon 714 (referenced to the human APP770 deduced translation as described in the “codon 714” definition), preferably isoleucine. Hence, an APP allele that encodes threonine at codon 714 is a “wild-type” APP allele. The sequence of APPT714I is depicted in SEQ ID NO: 1.

In a first embodiment, the present invention provides an isolated polynucleotide sequence that encodes an APP isoform or fragment that has an amino acid other than threonine at the amino acid position corresponding to amino acid residue position 714 of APP770, and in a specific embodiment of this invention this amino acid is an isoleucine.

In another embodiment the isolated polynucleotide sequence that encodes said mutant APP isoform or fragment is operably linked with a promotor.

In another embodiment, the present invention provides a human APP isoform or fragment, free from other human proteins, that has an amino acid other than threonine at the amino acid position corresponding to amino acid residue position 714 of APP770. As a not limited example of an expression system, baculovirus expression systems are useful for high level expression of heterologous genes in eukaryotic cells and Knops et al. (1991) J. Biol. Chem. 266(11): 7285, the contents of which are incorporated by reference herein, in its entirety) describe the expression of APP using said expression system.

In another embodiment, the present invention provides recombinant bacteria and cells, typically eukaryotic cells and preferably mammalian cells and more preferentially of the neural, glial, or astrocytic lineage, that have been transformed or transfected with a heterologous DNA sequence comprising the above described APP714 mutation, or have been derived from a transgenic non-human animal, wherein the cells express an APP isoform or fragment that has an amino acid other than threonine at the amino acid position corresponding to amino acid residue position 714 of APP770. In accordance with standard protocols, cultured human cells, either primary cultures or immortalized cell lines, may be transfected, either transiently or stably, with a mutant APP714 allele so that the cultured human cell expresses said mutant APP714 polypeptide.

In a specific embodiment, the cells can also be naturally derived. For this, blood samples or fibroblasts from the affected subject, diagnosed with the APP714 mutation, must be obtained in order to provide the necessary cells which can be permanently transformed into a lymphoblastoid cell line using, for example, Epstein-Barr virus. Once established, such cell lines can be grown continuously in suspension culture and may be used for a variety of in vitro experiments to study mutant APP714 expression and processing.

Since the APP714 mutation is dominant, an alternative method for constructing a cell line is to engineer genetically a mutated gene, or a portion thereof spanning codon 714, into an established (either stably or transiently) cell line of choice. Sisodia (1990) Science 248:492, the contents of which are incorporated by reference herein, in its entirety) has described the insertion of a normal APP gene, by transfection, into mammalian cells. Oltersdorf et al. (1990) J. Biol. Chem. 265:4492, the contents of which are incorporated by reference herein, in its entirety) describe the insertion of APP into immortalized eukaryotic cell lines.

In another embodiment, the present invention provides a transgenic non-human animal that carries in its somatic and germ cells at least one integrated copy of a human DNA sequence that encodes an APP isoform or fragment that has an amino acid other than threonine at the amino acid position corresponding to amino acid residue position 714 of APP770.

It is expected that the transgenic non-human animal, for example a transgenic mouse, will have a particular value because as with the human brain of an AD patient with the APP714 mutation, an exclusive deposition of Aβ₄₂ is expected. Overexpression of mutant APP with or without mutant presenilin 1 in mice shows a predominance of Aβ₄₀ deposition into dense-cored plaques. It also seems that in rodent brain Aβ₄₀, but not Aβ₄₂, is easily depositable as congophilic amyloid deposits that is important in the light that Aβ₄₂, but not Aβ₄₂, is proposed to be central to the aetiology of AD. In the current invention, both neuronal and non-neuronal recombinant cells for the mutant APP714, increases Aβ₄₂ secretion while decreasing Aβ₄₀. In brains of the affected patients there is almost no deposition of Aβ₄₀ or Aβ₃₉ plaques being composed entirely of Aβ₄₂. In conclusion, the novel transgenic mouse would mimic more the human disease phenotype of AD because in humans the deposition of Aβ₄₂ always supersedes the deposition of Aβ₄₀.

In a preferred example, it may be possible to excise the mutated APP714 gene for use in the creation of transgenic animals containing the mutated gene. In another example, an entire human APP714 allele may be cloned and isolated, either in parts or as a whole, in a cloning vector (e.g. cosmid or yeast or human artificial chromosome). The human variant APP714 gene, either in parts or in whole, may be transferred to a host non-human animal, such as a mouse or a rat. As a result of the transfer, the resultant transgenic non-human animal will preferably express one or more mutant APP714 polypeptides. Most preferably, a transgenic non-human animal of the invention will express one or more mutant APP714 polypeptides in a neuron-specific manner (Wirak et al. (1991) ^{EMBO J.} 10:289, the contents of which are incorporated by reference herein, in its entirety). This may be accomplished by transferring substantially the entire human APP gene (encoding a codon 714 mutant) including the 4.5 kilobase sequence that is adjacent to and upstream of the first major APP transcriptional start site.

Alternatively, one may design minigenes encoding variant APP codon 714 polypeptides. Such mini-genes may contain a cDNA sequence encoding a variant APP codon 714 polypeptide, preferably full-length, a combination of APP gene exons, or a combination thereof, linked to a downstream polyadenylation signal sequence and an upstream promoter (and preferably enhancer). Such a mini-gene construct will, when introduced into an appropriate transgenic host (e.g., mouse or rat), express an encoded variant APP codon 714 polypeptide, most preferably a variant APP codon 714 polypeptide that contains an isoleucine at codon 714 of APP770 or the corresponding position in an APP isoform or fragment.

Another approach to create transgenic animals is to target a mutation to the desired gene by homologous recombination in an embryonic stem (ES) cell line in vitro followed by microinjection of the modified ES cell line into a host blastocyst and subsequent incubation in a foster mother (see Frohman and Martin (1989) Cell 56:145, the contents of which are incorporated by reference herein, in its entirety). Alternatively, the technique of microinjection of the mutated gene, or a portion thereof, into a one-cell embryo followed by incubation in a foster mother can be used. Various uses of transgenic animals, particularly transgenic animals that express a wild-type APP isoform or fragment, are disclosed in Wirak et al. (1991) EMBO J., 10(2):289; Schilling et al. (1991) Gene 98(2):225; Quon et al. (1991) Nature 352:239; Wirak et al. (1991) Science 253:323; and Kawabata et al. (1991) Nature 354:476, the contents of each of which are incorporated by reference herein, in their entirety. Additional methods for producing transgenic animals are known in the art.

Alternatively, site-directed mutagenesis and/or gene conversion can be used to mutate a murine (or other non-human) APP gene allele, either endogenous or transfected, such that the mutated allele does not encode threonine at the codon position in the mouse APP gene that corresponds to codon 714 (of APP770) of the human APP gene (such position is readily identified by homology matching of the murine APP gene or APP protein to the human APP gene or APP770 protein). Preferably, such a mutated murine allele would encode isoleucine at the corresponding codon position.

The procedure for generating transgenic rats is similar to that of mice (Hammer et al., Cell 63; 1099-112 (1990), the contents of which are incorporated by reference herein, in its entirety). Thirty day-old female rats are given a subcutaneous injection of 20 IU of PMSG (0.1 cc) and 48 hours later each female placed with a proven male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are euthanized (CO₂ asphyxiation) and their oviducts removed, placed in DPBS (Dulbecco's phosphate buffered saline) with 0.5% BSA and the embryos collected. Cumulus cells surrounding the embryos are removed with hyaluronidase (1 mg/ml). The embryos are then washed and placed in EBSS (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, ip) and xylazine (5 mg/kg, ip). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10-12 embryos are transferred into each rat oviduct through the infundibulum. The incision is then closed with sutures, and the foster mothers are housed singly.

In another embodiment of the invention, transgenic cell lines transfected with a polynucleotide sequence, or an isoform or fragment thereof, encoding a codon 714 mutant of human amyloid precursor protein 770 can be used in a drug screening assay for the screening of molecules and monitoring the effectiveness of said molecules. Additionally, such model systems provide a tool for defining the underlying biochemistry of APP and β-amyloid metabolism, which thereby provides a basis for rational drug design. The specific effectiveness of a molecule is monitored by measuring the lower formation of the Aβ₄₂ peptide or N-truncated Aβ₄₂ peptide as compared with the amount of the Aβ₄₂ peptide or N-truncated Aβ₄₂ peptide without administering said molecule. Suitable molecules may be small molecules, biological polymers, such as polypeptides, polysaccharides, polynucleotides, and the like. Small molecules, e.g. small organic molecules, and other drug candidates can be obtained, for example, from combinatorial and natural product libraries. The test compounds will typically be administered to the culture medium at a concentration in the range from about 1 M to 1 mM, usually from about 10 μM to 1 mM.

Monitoring of the Aβ₄₂ peptide formation and/or truncated forms thereof can be measured using standard biochemical techniques, but preferentially with an ELISA assay. These assays may be performed using conventional techniques developed for these purposes in the course of screening.

To perform drug-screening assays, it is feasible to accommodate automation of the assay. Interaction (e.g., binding of) between the recombinant cells and the target molecules can be accomplished in any vessel suitable for containing the reactants. Examples of such vessels include microtiter plates, test tubes, and microcentrifuge tubes.

Also within the scope of the screening assay are oligonucleotide sequences that include anti-sense RNA and DNA molecules that function to inhibit the translation of specific mRNA targets in the recombinant cell lines. It is very well possible that such a target could for example be a β-secretase homologue or modulator of said β-secretase (homologue) or a protease involved in the generation of truncated forms of the Aβ₄₂ peptide such as N-truncated Aβ₄₂ peptides. For example, anti-sense RNA molecules may be generated in a library prepared by any method known in the art for the synthesis of gene libraries. Alternatively, the anti-sense sequences can be constructed in an anti-sense cDNA library under expression of an inducible or constitutive promoter.

In another embodiment the non-human transgenic animal, as described herein, can be analyzed for the formation and the inhibition of ‘cloudy’ diffuse plaques. The present mutation in APP714 leads to a severe AD pathology with unusual plaque composition, composed mainly of N-truncated Aβ₄₂, and morphology. It is found that the Aβ₄₀ form is nearly absent from amyloid deposits-in the human brain and the formation of typical dense-cored plaques is retarded. The predominant amyloid deposits are diffuse non-congophilic amyloid plaques in association with dystrophic neurites and reactive gliosis. In the present invention, novel pathological findings are presented that diffuse, non-congophilic (and thus nonfibrillar) Aβ plaques are pathogenic and cause AD. Furthermore, in the affected patients, it is shown that dystrophic neurites and a neurofibrillary pathology can develop in a plaque independent way. This demonstrates that nonfibrillar pre-amyloid deposits have the potential to cause neuronal toxicity and that the development of neuritic and amyloid pathology can be uncoupled at some stage of AD disease. In addition, the invention presents for the first time evidence, that N-truncated Aβ₄₂ plays a necessary and sufficient role to cause neuronal toxicity and cognitive decline in AD patients. In a specific embodiment, the non-human transgenic animal as described above can be used for the screening of molecules and monitoring said animal for the effect of said molecules on Aβ deposits in its brain. Preferentially molecules that are identified in the cellular screening assay and have an effect on the formation of N-truncated Aβ₄₂ and Aβ₄₂-formations are administered to the non-human transgenic animal. Monitoring of the transgenic animals can be done by pathological studies which include, but are not limited to, measuring the amount of plaques in the brain, measuring the amount of abnormally phosphorylated tau protein and/or the increase in the number of glial cells.

Another procedure that can be used is the measuring of a reduction in behavioural activity tests. Behavioral tests designed to assess learning and memory deficits are employed. An example of such as test is the Morris Water maze (Morris, Learn. Motivat. 12; 239-260 (1981), the contents of which are incorporated by reference herein, in its entirety). In this procedure, the animal is placed in a circular pool filled with water, with an escape platform submerged just below the surface of the water. A visible marker is placed on the platform so that the animal can find it by navigating toward a proximal visual cue. Alternatively, a more complex form of the test in which there are no formal cues to mark the platform's location will be given to the animals. In this form, the animal must learn the platform's location relative to distal visual cues.

The procedures applied to test transgenic mice are similar for transgenic rats.

Finally, in a last embodiment, the polynucleotide sequence that encodes an APP isoform or fragment that has an amino acid other than threonine, but preferably isoleucine, at the amino acid position corresponding to amino acid residue position 714 of APP770, which is a preferred substrate for γ-secretase, can be used for the identification of γ-secretase modulators and/or alternative processing enzymes which lead to N-truncated forms of Aβ₄₂ and C-truncated forms. Recently, compelling evidence was provided that the activity of γ-secretase is encoded by presenilins, which are a new class of aspartyl proteases (Li et al. (2000) Nature, 405, 689, the contents of which are incorporated by reference herein, in its entirety). The identification can be carried out preferentially in a double presenilin knock-out ES cell line in which no detectable γ-secretase activity is present anymore (Herreman A et al. (2000) Nat. Cell Biology 2, 461, the contents of which are incorporated by reference herein, in its entirety). Therefore, the double presenilin mutant ES cell line together with the APP714 mutation of the current invention is an excellent combinatorial tool to isolate and identify γ-secretase homologues, genetic modulators of γ-secretase activity and alternative processing enzymes of the APP protein. By “isolation” it is meant that standard molecular biology tools such as complementation, screening or selection cloning methods with a genomic or cDNA library are used to transfect the cells and to induce γ-secretase activity. Recombinant virus libraries, such as adenoviral, lentiviral or retroviral libraries could be used. The aforementioned methodology are only examples and do not rule out other possible approaches that could lead to potential candidates. Restoration of proteolytic activity can be followed by different means, to give only a few examples: ELISA assays or other assays measuring amyloid peptide production, or assays measuring Notch cleavage using luciferase reporter systems or other. To increase the sensitivity of such assays it can be considered to stably transfect the ES cells with cDNA's encoding APP714 mutant or other proteins and reporters useful for such assays.

The following examples more fully illustrate preferred features of the invention, but are not intended to limit the invention in any way. All of the starting materials and reagents disclosed below are known to those skilled in the art, and are available commercially or can be prepared using well-known techniques.

### EXAMPLES

APP T714I Mutation

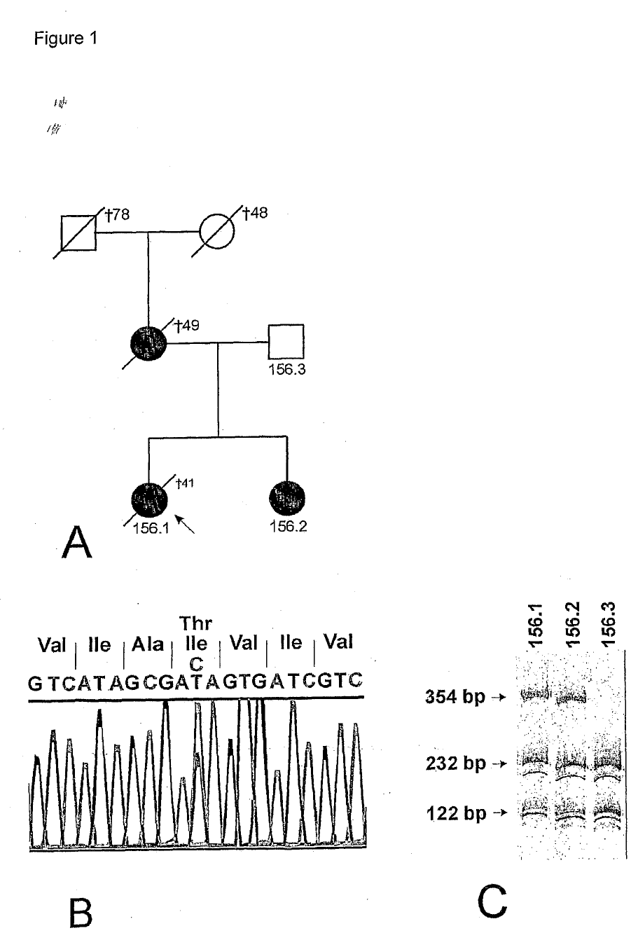

Family AD156 (FIG. 1A), an Austrian family consistent with autosomal dominant inheritance of early-onset AD, was referred for DNA diagnosis. The proband, her sister and their mother were diagnosed as probable AD according to NINCDS-ADRDA criteria at age 38, 38, and 44 years, respectively. However, signs of cognitive impairment and behavior disturbances were apparent several years earlier in all 3 patients suggestive of a mean onset age of ˜34 years in the family. Genomic DNA of the proband was examined for mutations in APP, PS1 and PS2. In exon 17 of APP (FIG. 1B), a heterozygous C to T transition was identified at position 2208 substituting Thr (T) at codon 714 by Ile (I) (T714I, numbering according to APP770 isoform). The mutation abolishes a TspRI restriction site, which was used to confirm the presence of the mutation in the proband (156.1) and her sister (156.2) (FIG. 1C). The mutation was absent in the father as well as in 50 healthy Austrian individuals. No other mutations were detected.

The Austrian T714I mutation is the first APP mutation reported to date that involves amino acid 43 of Aβ located directly at the γ₄₂-secretase cleavage site. The early onset age, as well as rapid progression of the disease, and early death, is comparable to AD caused by mutations in PSI.

Drastically Altered APP Processing in vitro

To understand the effect of the T714I mutation on the cleavage specificity of γ₄₂-secretase, we studied APP processing in non-neuronal and neuronal cells.

Human embryonic kidney (HEK) 293T cells were transiently transfected with the T714I APP cDNA and secreted Aβ₄₂ and Aβ₄₀ levels were measured in conditioned medium by enzyme linked immunosorbent assay (ELISA)(De Jonghe et al., 1999;De Strooper et al., 1998). Cells over-expressing wild type (WT) and London V717I (Goate et al., 1991) APP cDNA were used as controls. The T714I mutation increased Aβ₄₂ and at the same time decreased Aβ₄₀, resulting in a significantly increased Aβ₄₂/Aβ₄₀ ratio (p<0.001) that was 4 times higher than in WT APP. In the same experiment, V717I resulted in a 1.8 fold increased Aβ₁₋₄₂/Aβ₁₋₄₀ ratio solely due to increased Aβ₁₋₄₂, results that are comparable to previous published data (Suzuki et al., 1994).

We also measured (by ELISA) Aβ₁₋₄₂ and Aβ₁₋₄₀ in plasma of the patient (156.2), her unaffected father (156.3) and 5 unrelated age matched controls. The Aβ₁₋₄₂/Aβ₁₋₄₀ ratio was 2.5 fold increased compared to the unaffected father and 1.7 fold compared to the controls.

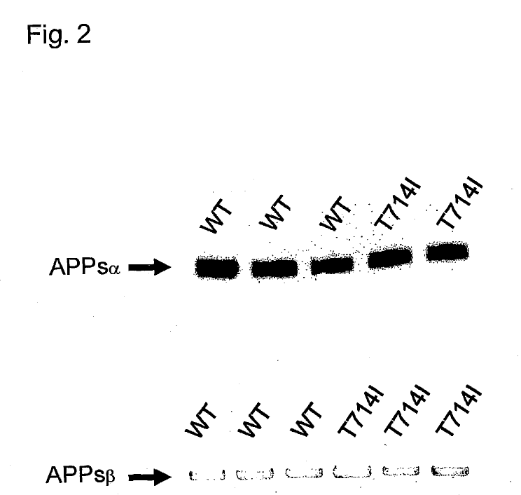

The conditioned medium of the T714I and WT APP transfected HEK293T cells was also analyzed by matrix-assisted laser desorption ionization time-of-flight (MALDI-TOF) mass spectrometry (FIG. 2)(Haass et al., 1992; Sisodia et al., 1990). This method allowed us to assess both full-length and N-truncated Aβ. Compared to WT, T714I showed a significant elevation of Aβ₁₋₄₂ by 6.4-fold (p<0.001), while at the same time Aβ₄₀ decreased significantly by 43% (p<0.001). This resulted in nearly equal levels of secreted Aβ₁₋₄₀ and Aβ₁₋₄₂ and an increased Aβ₁₋₄₂/Aβ₁₋₄₀ ratio by 10.8 fold. Similar effects were seen on p3 with equal levels of secreted Aβ₁₇₋₄₀ and Aβ₁₇₋₄₂ increasing Aβ₁₇₋₄₂/Aβ₁₇₋₄₀ ratio by 10.7 fold over WT.

One other pronounced affect of the T714I mutation is the increase of Aβ peptides ending at V39 and G38 irrespective of the N-terminal residue. Smaller effects were also seen for full-length Aβ and p3 peptides ending at G37. These peptides are not artificially produced by proteolysis of full-length Aβ as synthetic Aβ₁₋₄₀ and Aβ₁₋₄₂ peptides were not degraded when added to the medium of non-transfected cells (Wang et al., 1996). Despite a drastic decrease in Aβ₄₀, total Aβ, irrespective of its N- or C-terminal end (A_{X-X}) was increased by 2.5-fold for T714I. Compared to other APP mutations located close to the γ₄₂-secretase cleavage site (Ancolio et al., 1999; Cai et al., 1993; Goate et al., 1991; Suzuki et al., 1994), T714I has the highest increase in Aβ₄₂/Aβ₄₀.

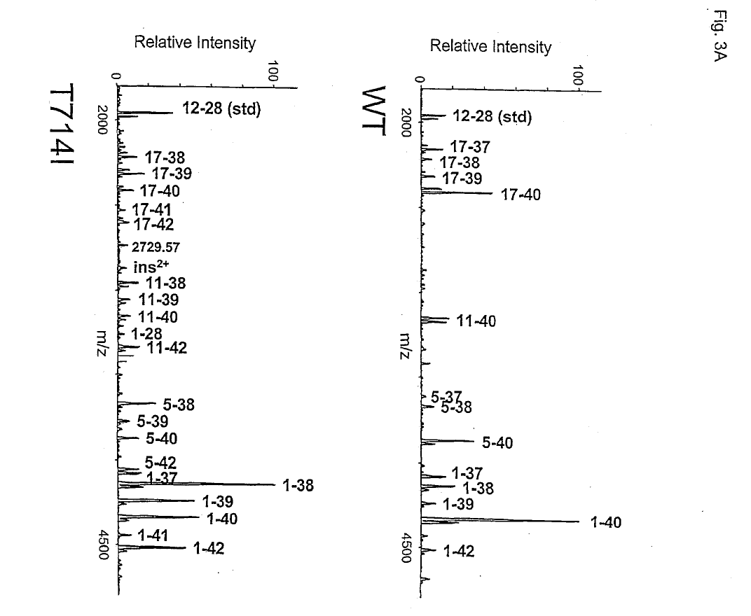

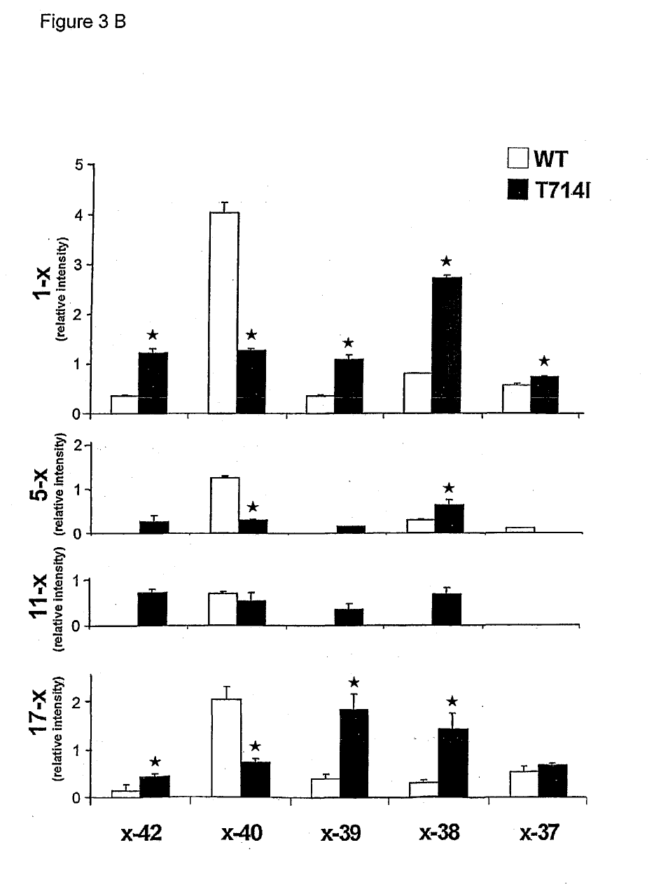

Interestingly, an in vitro decrease in Aβ₄₀ was also reported recently for the French 1715M mutation (Ancolio et al., 1999), one amino acid downstream of T714I. The mechanisms by which these mutations effect Aβ₄₀ and Aβ₄₂ secretion or lead to alternative cleavage to Aβ₃₈ or Aβ₃₉, is not yet understood. Distinct γ-secretases (Citron et al., 1996) might have different binding-affinities/cleavage-efficiencies to these mutated CTFs (Klafki et al., 1996) which in this region has an α-helix structure (Lichtenthaler et al., 1997), allowing amino acid residues three or four positions apart, to be spatially close. This might explain why T714I and V715F affect γ₄₀-secretase activity as well. To analyze whether the increase in total Aβ was due to an increased processing of CTFs by further action of γ-secretases, or alternatively to an increased trafficking of APP into cellular compartments where γ and β-secretases reside, we quantified APPs_{α} and APPs_{β}. No significant changes were noted (FIG. 3). Taken together with the increase in total Aβ (AB_{X-X}), the unaltered levels of APPs_{α} and APPs_{β} indicated that processing of CTFs could be a rate-limiting step in APP processing.

In a next step, we systematically analysed the effect of a series of clinical mutations near the γ-secretase cleavage site of APP in primary neurons. To this end, we expressed human WT APP or APP containing either the Austrian T714I (Kumar-Singh S (2000) Hum. Mol. Genet. 9, 2589, the contents of which are incorporated by reference herein, in its entirety), French V715M (Ancolio K (1999) PNAS 96, 4119, the contents of which are incorporated by reference herein, in its entirety), German V715A (Van Broeckhoven, unpublished data), Florida 1716V (Eckman C (1997) Hum. Mol. Genet. 6, 2087, the contents of which are incorporated by reference herein, in its entirety), Indiana V717L (Murrell JR (2000) Arch. Neurol. 57, 885, the contents of which are incorporated by reference herein, in its entirety) or London V717I (Goate A (1991) Nature 349, 704, the contents of which are incorporated by reference herein, in its entirety) mutation in primary cultures of neurons. We specifically immunoprecipitated Aβ₄₀^{˜} or Aβ₄₂ from the conditioned medium using antibody FCA3340 or antibody FCA3542 respectively (Barelli H (1997) Mol. Med. 3, 695, the contents of which are incorporated by reference herein, in its entirety). All APP C-terminal mutations with the exception of V715M, increased Aβ₁₋₄₂ secretion. The increase in Aβ₄₂ ranged from 1.54 times for T714I to 2.71 times for V717L. This was accompanied by an increase in the N-truncated Aβ-isoforms ending at residue 42 (Aβₓ₋₄₂). These isoforms run in the gels as doublet bands, and are presumably generated by alternative β-secretase activity at amino acid residue 11 or by α-secretase activity at amino acid residue 17 of the Aβ sequence. Aβ₁₋₄₀, on the other hand, is decreased with most mutations. The T714I, V715M and V715A mutations reduce Aβ₁₋₄₀ secretion most drastically to 20%, 30% and 55% of the wild type levels, respectively. The V717I and V717L mutations affect Aβ₄₀ secretion to a lesser extent and the 1716V mutation has apparently no effect on Aβ₄₀ secretion. The secretion of N-truncated Aβ₄₀ isoforms (Aαₓ₋₄₀) was decreased to an extent comparable to Aβ₁₋₄₀. Hence, when the Aβ₁₋₄₂/Aβ₁₋₄₀ ratio is compared for each mutation to WT APP (arbitrarily set equal to 1), this ratio is increased in all APP mutations. This increase ranges from 1.89 times for V717I to 8.20 times for T714I. Moreover, the Aβ₁₋₄₂/Aβ₁₋₄₀ ratio correlates inversely (r=−0.86) with the mean onset age for the different mutations.

Unusual Plaque Morphology and Composition

Neuropathological examination of the proband (156.1) showed extensive neuronal loss accompanied by diffuse gliosis, amyloid plaques and neurofibrillary tangles confirming the diagnosis of AD. We performed an in-depth analysis by histo- and immunohistochemistry on serial sections from 3 select brain areas, viz. hippocampus including entorhinal cortex (ERC), and temporal and frontal cortices. Immunostaining with an antibody recognizing both full-length Aβ and p3 (mAb 4G8), remarkably stained a huge plaque load predominantly as ‘cloud-like’ diffuse plaques that sometimes enclosed a central lacuna. In the molecular layer of the dentate gyrus, the amyloid plaques had the same non-neuritic ‘cotton wool’ plaques as described for PSI Δ9 patients (Crook et al., 1998). We determined the fibrillar nature of the amyloid plaques by Congo red and thioflavin staining. Dense-cored plaques were congophilic and fluorescent with thioflavin, while the majority of the diffuse plaques were non-congophilic. In the few diffuse plaques faintly positive for thioflavin, the fibrillar amyloid occupied only a small proportion of total amyloid recognized immunohistochemically. In the layer III of ERC, the amyloid plaques were congophilic while in its superficial and deep layers, the diffuse plaques were again nonfibrillar. A strong neuritic pathology was evident in CA1 and subicular fields of hippocampus and ERC. Neurites accumulated hyperphosphorylated tau (AT8), ubiquitin, and APP, irrespective of plaque pathology. Using endothelial cell markers (CD31 and CD34) with amyloid staining, senile plaque cores but not the diffuse plaques were closely associated with blood vessels. This feature was most remarkable in the end plate region where all fibrillar amyloid deposits were associated with blood vessels. Next, we examined whether in vivo the mutation had similar effects on APP processing. Reactivity to a C-terminal Aβ₄₀ specific monoclonal antibody (mAb), JRF/cAb40/10, was completely absent in both blood vessel walls and amyloid plaques in the isocortex, while a faint immunoreactivity was occasionally present in the endplate region of the hippocampus (2-3 amyloid deposits per 10 fields of 0.7 mm² each).

By contrast, a C-terminal Aβ₄₂ specific mAb, JRF/cAb42/12, imparted a strong reactivity to all amyloid deposits including diffuse plaques, blood vessel walls and ‘infrequent’ dense-cored plaques. Sections were stained with 4G8 and compared to immunoreactivity for antibodies specific for Aβ₄₀ and Aβ₄₂ by image analysis. In hippocampus, Aβ₄₀ constituted only 1-7% of amyloid deposits while in neocortex was completely absent. Multi-spectral analysis by confocal laser scanning microscope (CLSM) also demonstrated the absence of Aβ₄₀ and a full histochemical overlap for 4G8 and Aβ₄₂ specific antibody. These observations were confinned using other Aβ₄₀ specific (FCA3340; Clements et al., 1993, and R209; Mehta et al., 2000) and Aβ₄₂ specific antibodies (21F12, Johnson-Wood et al., 1997; FCA3542, Clements et al., 1993; R226, Mehta et al., 2000). We also checked for presence of Aβ ending at Val39 (Aβ₃₉) and Thr43 (Aβ₄₃), however, neither of these peptides were significantly deposited in APP714 brain. We studied the N-truncation-of Aβ in these amyloid deposits and confirmed whether the diffuse plaques were capable of inciting any glial reaction. Reactivity for a panel of antibodies against N-terminal Aβ (6E10, 6F/3D, and JRF/ABN/11) when compared to 4G8 reactivity, indicated that diffuse plaques constituted N-truncated Aβ while full-length Aβ was confined to blood vessel walls, dense amyloid cores and amyloid plaques in ERC layer III and remarkably negative for deep cortical layers. Abundant glial and inflammatory pathology was noted in association with diffuse as well as compact plaques using astroglial (GFAP), microglial (CD68, HLA-DR), and complement (Clq) markers. Microglia were present in the vicinity of compact plaques, although an intense glial activation was noted in many regions without plaque pathology as in white matter.

Generation of Transgenic Mice

Transgenic mice were generated. The sequence of a construct where the mutant APP714 is under control of the platelet derived growth factor (PDGF) promoter is depicted in SEQ ID NO: 2. A linearized construct such as SEQ ID NO:2, is purified and microinjected into 1.5 days old pre-nuclear embryos isolated from superovulated females of four kinds of Fl hybrid strains: C57/B1/6 X CBA, C57/B/6 X DBA, C57/B1/6 X D2, C57/B1/6 X C3H.

Materials and Methods

AD Diagnosis

Patients in family AD156 were diagnosed with AD based on neurological examination, neuropsychological testing, neuro-imaging, and neuropathology (Kleinert et al., in preparation). The mother was diagnosed at age 44 years. She had progressive memory problems and was disoriented in time. EEG showed moderate but generalized unspecific changes while CT showed brain atrophy. The proband as well as her sister had a neurological examination at age 38 years. They both suffered from severe depression. Mini metal state examination (MMSE) confirmed the presence of dementia. SPECT showed clear hypoactivity while CT confirmed the presence of brain atrophy. In both sisters, the dementia was rapidly progressing as measured repeatedly by MMSE. For example, at age 39 years the proband scored 18/30 and her sister 11/30, at age 40 years the scores had already dropped to respectively 10/30 and 3/30. The actual age of onset of the symptoms was several years earlier according to the neurologists who treated the patients. Onset age in the mother was estimated 5-7 years and in the daughters 4-5 years earlier. Therefore, mean onset age in family AD 156 was estimated at ˜34 years. The APOE genotype of the proband was E3E3, that of the sister E2E3. The APOE genotype of the mother E2E3 was inferred from that of the father and siblings. The proband died at age 41 years and had brain autopsy, the sister is still alive at age 42 years. Macroscopic examination of the brain showed gross atrophy weighing ˜1000 g. Sections derived from the fore-, mid- and hindbrain were stained with haematoxylin-eosin (HE), Nissl, Congo red, and modified Bielshowsky. A definite diagnosis of presenile AD was made.

Genetic Analysis

Exons 16 and 17 of APP were PCR amplified from genomic DNA of patient 156.1 using published primer sets and PCR conditions (Bakker et al., 1991) and PCR fragments were sequenced using the ‘Ready Reaction Rhodamine Dye Terminator Cycle Sequencing’ kit (Applied Biosystems, Foster City, USA). The products were analyzed on an ABI310 capillary DNA sequencer (Applied Biosystems). The APP T714I mutation was analyzed by TspRI digestion of PCR amplified APP exon 17. Wild-type (WT) fragments of 354 bp are cut into 2 fragments of 232 and 122 bp respectively, while the T714I mutant fragments are not cut (FIG. 1). All coding exons of PS1 and PS2 were PCR amplified from genomic DNA using published primer sets (Cruts et al., 1998) and screened for mutations by single strand conformational polymorphism (SSCP) analysis as described (Cruts et al., 1998). APOE genotype was determined as described (van Duijn et al., 1994; Wenhan et al., 1991).

In vitro Mutagenesis

Site-directed mutagenesis was performed on WT APP₆₉₅ cDNA cloned in pCDNA3 (Hendriks et al., 1995) using the ‘QuikChange site-directed mutagenesis’ system (Stratagene, La Jolla, Calif., USA). Primers app714s (5′-CGGTGTTGTCATAGCGATAGTGAT-CGTCATCACC-3′ SEQ ID NO: 4) and app714 as (5′-GGTGATGACGATCACTATCGCTAT-GACAACACCG-3′ SEQ ID NO: 5) were used to insert the APP T714I mutation into the construct. The sequence of the constructs was confirmed by direct PCR sequencing of the insertion fragment using the ‘Taq dye terminator sequenase II sequencing’ kit (Applied Biosystems, Foster City, USA). The products were analyzed on an ABI373 automated DNA sequencer (Applied Biosystems). Mutant APP V717I were constructed as described previously (Hendriks et al., 1995).

cDNA Transfection

Human embryonic kidney (HEK-293T) cells were transiently transfected with pCDNA3 vector containing the T714I, V717I or WT APP₆₉₅ cDNA constructs using Fugene (Roche Diagnostics) according to the manufacturer's procedures. The presence of the constructs in the cells was confirmed by Western Blotting. To normalize for APP expression, cells were lyzed in 300 l RIPA buffer (50 mM Tris, pH 8.0, 150 mM NaCl, 1% NP-40, 0.5% sodium deoxycholate, 0.1% SDS+complete protease inhibitors). A dilution series of a 5 μl aliquot was separated on a 4-12% NuPAGE polyacrylamide gel. Proteins were blotted on a PVDF membrane and immunodetection was performed with antibody 10B4 (Senetek) using the Western Star Chemiluminescence system (Tropix). The full-length APP immunoreactive band was quantified using the NIH Image software package.

Aβ ELISA

HEK293T cells were transfected in triplicate with WT or T714I APP cDNA in a 6 well plate. One day after transfection, 1 ml OPTIMEM medium without additives was added to the HEK-293T cells and conditioned for 24 hrs. Medium was collected and pooled from 6 transfections. A 1 ml aliquot was used for Aβ3 ELISA. Aβ₄₂ concentrations were measured in the conditioned media by ELISA, using a prototype version of the INNOTEST β-amyloid₁₋₄₂ HS ELISA detecting Aβ₄₂ peptide (De Strooper et al., 1998). Aβ₄₀ was measured by ELISA using rabbit antiserum R209 (Mehta et al., 2000) as capturing antibody and biotinylated 3D6 (Johnson-Wood et al., 1997) as detector antibody as described (De Jonghe et al., 1999; De Strooper et al., 1998). Each experiment was performed in triplicate and the results were averaged. A two-tailed unpaired t-test was used to compare the mean level of Aβ produced by the WT and mutant transfectants.

Mass spectrometric Aβ Analysis

In a second aliquot of supernatant, collected as described above, proteinase inhibitors (2 mM EDTA-Na, 10 μM leupeptin, 1 μM pepstatin A, 1 mM PMSF, 0.1 mM TLCK, 0.2 mM TPCK) were added. Aβ peptides were analyzed by immunoprecipitation/mass spectrometric Aβ assay (IP/MS) as described previously (Wang et al., 1996). The Aβ peptides were immunoprecipitated from 1.0 ml of conditioned media using mAb 4G8 (Senetek, Maryland Heights, Mo.) and protein G Plus/Protein A-agarose beads (Oncogene Science, Inc., Cambridge, Mass.) and analyzed using a MALDI-TOF mass spectrometer (Voyager-DE STR BioSpectrometry Workstation, PE/PerSeptive Biosystem). Each mass spectrum was averaged from 256 measurements and calibrated by using bovine insulin as internal mass calibrant. For comparing the peptide levels in the conditioned media, synthetic Aβ (12-28) peptide (10 nM) was used as internal standard and the relative peak intensity was used. Both ELISA and MALDI-TOF mass spectrometric analysis were performed by experimenters ‘blinded’ to sample identity.

Quantification of APPs_{α} and APPs_{β}

5 μl conditioned medium was separated on a 4-12% NuPage gel (Novex). Proteins were transferred to a PVDF membrane, the membrane was blocked in PBS+0.2% I-block+0.1% TWEEN-20, incubated over night at 4° C. with primary antibodies 6E10 diluted 1:2000 (for APPs_{α}) or 53/4 diluted 1/500 (for APPs_{β}), incubated with alkaline phosphatase labeled secondary antibody (1/4000 diluted), and detected with either Western Star chemiluminescent reagent (Tropix) or ECL (Amersham).

Immunohistochemistry

MAb JRF/cAb40/10 and JRF/cAb42/12 specific for the C-terminus of Aβ₄₀ and Aβ₄₂, respectively, were raised by immunizing mice with synthetic peptides corresponding to Aβ3 residues 36-40 (VGGVV SEQ ID NO: 6) or residues 33-42 (GLMVGGVVIA SEQ ID NO: 7) (Mercken et al., unpublished data). Specificity of the Aβ₄₀ and Aβ₄₂ mAbs was validated by ELISA and Western blotting showing no cross reactivity. Similarly, mAb JRF/AβN/11 specific for N-terminus of Aβ was raised against Aβ residues 1-7 (DAEFRHD (SEQ ID NO: 8) and recognizes full-length Aβ. In addition for immunohistochemistry, the following antibodies were used: mAb 6E10 (Senetek; raised against Aβ 1-17, recognizes Aβ 5-13, Rong Wang, personal communication), mAb 4G8 (Senetek; Aβ residues 17-24), 6F3D (Dako; raised against Aβ residues 7-17), mAb 21F12 (for Aβ₄₂, Innogenetics, Belgium), rabbit antisera FCA3542 and FCA3340 (Barelli et al., 1997), rabbit Aβ₄₀ antisera R209 (Mehta et al., 2000) and Aβ₄₂ R226 (Mehta et al., 2000), rabbit anti-Aβ₁₋₄₀ (Sigma, St. Louis, Mo., USA), rabbit anti-Aβ3₃₉ and anti-Aβ₄₃ (T. Saido, Laboratory for Proteolytic Neuroscience, RIKEN Brain Science Institute, Wako-shi 351-0198 Japan), mAb 22C11 (N-terminus APP; Roche), mAb AT8 (abnormally phosphorylated tau; Innogenetics, Ghent B.E.), and anti-glial fibrillary acidic protein (GFAP; Dako, Glostrup, Denmark), CD68 (macrophage; Dako), rabbit anti-ubiquitin (Dako), Clq complement (Dako), HLA-DR (HLA-DP,DQ,DR; Dako), CD31(Dako), and CD34 (QBEnd; Dako). Antigen retrieval for Aβ immunohistochemistry was performed on sections treated with 98% formic acid for 10 min, and for other antibodies as recommended by the supplier. Staining for single antigen was performed using streptavidin-biotin-horse radish peroxidase (ABC/HRP) or peroxidase-anti-peroxidase (PAP), utilizing 3′3′diaminobenzidine (DAB) as a chromogen as described elsewhere (Kumar-Singh et al., 1997). Immunohistochemistry involving detection of more than one antigen was done using species-specific or IgG subtype-specific secondary antibodies conjugated directly with biotin, HRP, alkaline phosphotase or Galactosidase (Southern Biotechnology, Birmingham, Ala., USA). This was followed by color development using one of the following chromogens (Roche): DAB, 3-amino-9-ethylcarbazole (AEC), Fast-red, 5-bromo-4-chloro-3indolul phosphate/nitroblue tetrazolium solution (BCIP/NBT) or 5-Bromo-4-chloro-3-indolyl-β-D-galactopyranoside (X-gal). For Aβ₄₀ immunohisto-chemistry, a sensitive tyramide amplification system (NEN, MA, USA) was utilized.

Densitometric Analysis

Densitometric analysis was performed for staining 5 μm thick serial sections stained with 21F12, JRF/cAb42/12, and FCA3542 (for Aβ₄₂), JRF/cAb40/10, R209 (Mchta et al., 2000), and FCA3340 (for Aβ₄₀), and 4G8, was performed using the Vidas image analysis system (Kontron) and the obtained results were compared with staining of similar brain regions of patients with sporadic AD cases and PS1 (1143T) related familial AD. Pixels representing the immunocytochemical stain were counted to calculate the size of each plaque. Also, the relative area occupied by Aβ staining in five fields from hippocampus or ERC with most intense staining and envisaging an area of 0.7 mm², was determined as described elsewhere (Kumar-Singh et al., 1997).

Fluorescent microscopy

For multiple labeling on a CLSM, 10 μm sections were incubated overnight with mAb 21F12 and a JRF/cAb42/12, washed and labeled with an anti-mouse TRITC conjugated and an anti-rabbit FITC antibody (Molecular Probes, Eugene, Oreg., USA). Images were acquired with a Zeiss CLM-410 using either 488-nm-line of argon single laser or 632-nm helium-neon double laser for excitation. One μm thick consecutive optical slices were captured for both fluorochromes separately, and the relative Aβ₄₂/Aβ₄₀ content and ratio in amyloid plaques were determined.

Preparation of Recombinant SFV Virus

The Austrian T714I, German V715A, French V715M, Florida 1716V, and Indiana V717L mutations were introduced in human WT APP cDNA (695 isoform) by site-directed mutagenesis using appropriate oligonucleotides and the Quick-change mutagenesis system (Stratagene, La Jolla, Calif.). The mutant APP cDNA was subsequently cloned into the Smal-site of pSFV-1 (Gibco BRL, Bethesda, Md.). Recombinant Semliki Forest Viruses were produced as described (Annaert WG et al. (1999) J. Cell Biol. 147, 277, which is incorporated herein by reference in its entirety).

Primary Neuronal Cultures

Primary cortical neurons were isolated from E14 embryonic mice as described (Annaert W G et al. (1999) J. Cell Biol. 147, 277). Briefly, after dissection of the brain and dissociation of the cells by trypsinisation, cells were plated on poly-lysine coated dishes and incubated in neurobasal medium+B27 supplement. Proliferation of non-neuronal cells was prevented by adding 5 μM cytosine arabinoside.

Infection of Primary Neurons

Recombinant SFV was diluted 1:10 in culture medium and added to 3 to 5 day-old primary cortical neurons. After adsorption for one hour, the viral vector containing solution was replaced with normal culture medium and transduction continued for 2 hours. Medium was then replaced by methionine-free medium containing 100 μCi ³⁵S-methionine (ICN, Irvine, Calif.). After 4 hours of metabolic labelling, culture supernatants were collected, cells were washed in PBS and finally lysed in DIP buffer (20 mM Tris-HCI, pH 7.4, 150 mM NaCl, 1% Triton X-100, 1% sodium deoxycholate, 0,1% SDS), containing protease inhibitors (5 mM EDTA, trasylol, 1 μg/ml pepstatin).

### REFERENCES

Each of the following references is incorporated in its entirety, as fully set forth herein.

1. Glenner, G. G. & Wong, C. W. Alzheimer's disease: Initial report of the purification and characterization of a novel cerebrovascular amyloid protein. Biochem. Biophys. Res. Commun. 122, 885-890 (1984).

2. Masters, C. L. et al. Amyloid plaque core protein in Alzheimer disease and Down syndrome. Proc. Natl. Acad. Sci. USA 82, 4245-4249 (1985).

3. Kang, J. et al. The precursor of Alzheimer's disease amyloid A4 protein resembles a cell-surface receptor. Nature 325, 733-736 (1987).

4. Selkoe, D. J. Alzheimer's disease: genotypes, phenotypes, and treatments. Science 275, 630-631 (1997).

5. Vassar, R. et al. Beta-secretase cleavage of Alzheimer's amyloid precursor protein by the transmembrane aspartic protease BACE [see comments]. Science 286, 735-741 (1999).

6. Selkoe, D. J. The cell biology of beta-amyloid precursor protein and presenilin in Alzheimer's disease. Trends. Cell Biol. 8, 447-453 (1998).

7. Wang, R., Sweeney, D., Gandy, S. E. & Sisodia, S. S. The profile of soluble amyloid beta protein in cultured cell media. Detection and quantification of amyloid beta protein and variants by immunoprecipitation-mass spectrometry. J. Biol. Chem. 271, 31894-31902 (1996).

8. Cruts, M. & Van Broeckhoven, C. Presenilin mutations in Alzheimer's disease. Hum. Mutat. 11, 183-190 (1998).

9. http://molgen-www.uia.ac.be/ADMutations http://www.alzforum.org/members/resources/app_mutations/app_table.html.

10. Mullan, M. et al. A pathogenic mutation for probable Alzheimer's disease in the APP gene at the N-terminus of beta-amyloid. Nat. Genet. 1, 345-347 (1992).

11. Citron, M. et al. Mutation of the alpha-amyloid precursor protein in familial Alzheimer's disease increases alpha-protein production. Nature 360, 672-674 (1992).

12. Cai, X.-D., Golde, T. E. & Younkin, S. G. Release of excess amyloid alpha protein from a mutant amyloid alpha protein precursor. Science 259, 514-516 (1993).

13. Suzuki,N. et al. An increased percentage of long amyloid beta protein secreted by familial amyloid beta protein precursor (beta APP717) mutants. Science 264, 1336-1340 (1994).

14. Goate, A. et al. Segregation of a missense mutation in the amyloid precursor protein gene with familial Alzheimer's disease. Nature 349, 704-706 (1991).

15. Ancolio, K. et al. Unusual phenotypic alteration of beta amyloid precursor protein (betaAPP) maturation by a new val-715->Met betaAPP-770 mutation responsible for probable early-onset Alzheimer's disease. Proc. Nat/. Acad. Sci. U. S. A. 96, 4119-4124 (1999).

16. Hardy, J., Duff, K., Gwinn Hardy, K., Perez-Tur, J. & Hutton, M. Genetic dissection of Alzheimer's disease and related dementias: amyloid and its relationship to tau. Nature Neuroscience 1, 355-358 (1998).

17. Mori, H., Takio, K., Ogawara, M. & Selkoe, D. J. Mass spectrometry of purified amyloid beta protein in Alzheimer's disease. J Biol. Chem. 267, 17082-17086 (1992).

18. Dickson, D. W. The pathogenesis of senile plaques. J. Neuropathol. Exp. Neurol. 56, 321-339 (1997).

19. Iwatsubo, T. et al. Visualization of A beta 42(43) and A beta 40 in senile plaques with end-specific A beta monoclonals: evidence that an initially deposited species is A beta 42(43). Neuron 13, 45-53 (1994).

20. Iwatsubo, T., Mann, D. M., Odaka, A., Suzuki, N. & Ihara, Y. Amyloid beta protein (A beta) deposition: A beta 42(43) precedes A beta 40 in Down syndrome [see comments]. Ann. Neurol. 37, 294-299 (1995).

21. Mann, D. M. et al. Predominant deposition of amyloid-beta 42(43) in plaques in cases of Alzheimer's disease and hereditary cerebral hemorrhage associated with mutations in the amyloid precursor protein gene. Am. J. Pathol. 148, 1257-1266 (1996).

22. Fukumoto, H. et al. Amyloid beta protein deposition in normal aging has the same characteristics as that in Alzheimer's disease. Predominance of A beta 42(43) and association of A beta 40 with cored plaques. Am. J. Pathol. 148, 259-265 (1996).

23. Joachim, C. L. & Selkoe, D. J. The seminal role of beta-amyloid in the pathogenesis of Alzheimer disease. Alzheimer. Dis. Assoc. Disord. 6, 7-34 (1992).

24. Mattson, M. P. Cellular actions of beta-amyloid precursor protein and its soluble and fibrillogenic derivatives. Physiol. Rev. 77, 1081-1132 (1997).

25. Geula, C. et al. Aging renders the brain vulnerable to amyloid beta-protein neurotoxicity. Nat. Med. 4, 827-831 (1998).

26. McKee, A. C., Kowall, N. W., Schumacher, J. S. & Beal, M. F. The neurotoxicity of amyloid beta protein in aged primates. Amyloid. 5, 1-9 (1998).

27. Gowing, E. et al. Chemical characterization of A beta 17-42 peptide, a component of diffuse amyloid deposits of Alzheimer disease. J. Biol. Chem. 269, 10987-10990 (1994).

28. Weisgraber, K. H. & Mahley, R. W. Human apolipoprotein E: the Alzheimer's disease connection. FASEB J. 10, 1485-1494 (1996).

29. Velazquez, P., Cribbs, D. H., Poulos, T. L. & Tenner, A. J. Aspartate residue 7 in amyloid beta-protein is critical for classical complement pathway activation: implications for Alzheimer's disease pathogenesis. Nat. Med. 3, 77-79 (1997).

30. De Jonghe, C. et al. Aberrant splicing in the presenilin-1 intron 4 mutation causes presenile Alzheimer's disease by increased Abeta42 secretion. Hum. Mol Genet. 8, 1529-1540 (1999).

31. Clements, A., Walsh, D. M., Williams, C. H. & Allsop, D. Effects of the mutations Glu22 to Gln and Ala21 to Gly on the aggregation of a synthetic fragment of the Alzheimer's amyloid beta/A4 peptide. Neurosci. Lett. 161, 17-20 (1993).

32. Lichtenthaler, S. F. et al. Mechanism of the cleavage specificity of Alzheimer's disease gamma-secretase identified by phenylalanine-scanning mutagenesis of the transmembrane domain of the amyloid precursor protein. Proc. Natl. Acad. Sci. U.S. A. 96, 3053-3058 (1999).

33. Citron, M. et al. Evidence that the 42- and 40-amino acid forms of amyloid beta protein are generated from the beta-amyloid precursor protein by different protease activities. Proc. Natl. Acad. Sci. U.S. A. 93, 13170-13175 (1996).

34. Klafki, H., Abramowski, D., Swoboda, R., Paganetti, P. A. & Staufenbiel, M. The carboxyl termini of beta-amyloid peptides 1-40 and 1-42 are generated by distinct gamma-secretase activities. J Biol. Chem. 271, 28655-28659 (1996).

35. Yamazaki, T., Haass, C., Saido, T. C., Omura, S. & Ihara, Y. Specific increase in amyloid beta-protein 42 secretion ratio by calpain inhibition. Biochemistry 36, 8377-8383 (1997).

36. Lichtenthaler, S. F., Ida, N., Multhaup, G., Masters, C. L. & Beyreuther, K. Mutations in the transmembrane domain of APP altering gamma-secretase specificity. Biochemistry 36, 15396-15403 (1997).

37. Kuo, Y. M. et al. Water-soluble Abeta (N-40, N-42) oligomers in normal and Alzheimer disease brains. .J Biol. Chem. 271, 4077-4081 (1996).

38. Watson, D. J., Selkoe, D. J. & Teplow, D. B. Effects of the amyloid precursor protein Glu693-->Gln ‘Dutch’ mutation on the production and stability of amyloid beta-protein. Biochem. J. 340, 703-709 (1999).

39. Jarrett, J. T., Berger, E. P. & Lansbury, P. T. Jr. The carboxy terminus of the -amyloid protein is critical for the seeding of amyloid formation: Implications for the pathogenesis of Alzheimer's disease. Biochemistry 32, 4693-4697 (1993).

40. Shin, R. W. et al. Amyloid beta-protein (Abeta) 1-40 but not Abetal-42 contributes to the experimental formation of Alzheimer disease amyloid fibrils in rat brain. J. Neurosci. 17, 8187-8193 (1997).

41. McGowan, E. et al. Amyloid phenotype characterization of transgenic mice overexpressing both mutant amyloid precursor protein and mutant presenilin 1 transgenes. Neurobiol. Dis. 6, 231-244 (1999).

42. Mori, H., Takio, K., Ogawara, M. & Selkoe, D. J. Mass spectrometry of purified amyloid beta protein in Alzheimer's disease. J. Biol. Chem. 267, 17082-17086 (1992).

43. Tamaoka,A. et al. Amyloid beta protein 1-42/43 (A beta 1-42/43) in cerebellar diffuse plaques: enzyme-linked immunosorbent assay and immunocytochemical study. Brain Res. 679, 151-156 (1995).

44. Crook, R. et al. A variant of Alzheimer's disease with spastic paraparesis and unusual plaques due to deletion of exon 9 of presenilin 1. Nat. Med. 4, 452-455 (1998).

45. Verkkoniemi, A. et al. Variant Alzheimer's disease with spastic paraparesis: clinical characterization [In Process Citation]. Neurology Mar. 14, 2000.;54. (5.).1103. -9. 54, 1103-1109.

46. Hartley, D. M. et al. Protofibrillar intermediates of amyloid beta-protein induce acute electrophysiological changes and progressive neurotoxicity in cortical neurons. J. Neurosci. 19, 8876-8884 (1999).

47. Bakker, E. et al. DNA diagnosis for hereditary cerebral hemorrhage with amyloidosis (Dutch type). Am. J. Hum. Genet. 49, 518-521 (1991).

48. Cruts, M. et al. Estimation of the genetic contribution of presenilin-1 and -2 mutations in a population-based study of presenile Alzheimer disease. Hum. Mol. Genet. 7, 43-51 (1998).

49. Wenhan, P. R., Price, W. H. & Blundell, G. Apolipoprotein E genotyping by one-stage PCR. Lancet 337, 1158-1159 (1991).

50. van Duijn, C. M. et al. Apolipoprotein E4 allele in a population-based study of early-onset Alzheimer's disease. Nat. Genet. 7, 74-78 (1994).

51. Hendriks, L., Cras, P., Martin, J. J. & Van Broeckhoven C. Alzheimer's Disease: Lessons from Cell Biology. Kosik, K. S. (ed.), pp. 37-48 (Springer-Verlag, Berlin, Heidelberg, 1995).

52. De Strooper, B. et al. Deficiency of presenilin-1 inhibits the normal cleavage of amyloid precursor protein [see comments]. Nature 391, 387-390 (1998).

53. Johnson-Wood, K. et al. Amyloid precursor protein processing and A beta42 deposition in a transgenic mouse model of Alzheimer disease. Proc. Natl. Acad. Sci. U.S. A. 94, 1550-1555 (1997).

54. Barelli, H. et al. Characterization of new polyclonal antibodies specific for 40 and 42 amino acid-long amyloid beta peptides: their use to examine the cell biology of presenilins and the immunohistochemistry of sporadic Alzheimer's disease and cerebral amyloid angiopathy cases. Mol Med 3, 695-707 (1997).

55. Kumar-Singh, S. et al. Evaluation of tumour angiogenesis as a prognostic marker in malignant mesothelioma. J Pathol 182, 211-216 (1997).

We claim:

1. An isolated polynucleotide sequence, or an isoform or fragment thereof, encoding a codon 714 mutant of human amyloid precursor protein 770.

2. The isolated polynucleotide sequence of claim 1, wherein the amino acid at the position encoded by codon 714 is an isoleucine.

3. The isolated polynucleotide of any one of claims 1 to 2, wherein the polynucleotide sequence is a cDNA.

4. The polynucleotide of any one of claims 1-3, wherein said polynucleotide sequence is operably linked with a promoter.

5. The polynucleotide of any one of claims 1-4, wherein the polynucleotide incorporates at least one substitution other than at the position encoded by codon 714.

6. A protein encoded by the polynucleotide according to any one of claims 1-5.

7. A transgenic cell comprising the polynucleotide according to any one of claims 1-5.

8. The transgenic cell of claim 7, wherein said transgenic cell is selected from the group consisting of a eukaryotic primary cell, an embryonic stem cell line, and an immortalized cell line.

9. The transgenic cell of claim 7, wherein said transgenic cell is a bacterium.

10. The transgenic cell of any one of claims 7 or 8, wherein said polynucleotide is integrated into the cell's genome.

11. A non-human transgenic animal of which somatic and germ cells comprise the polynucleotide of any one of claims 1-5.

12. A method of screening a molecule which is able to reduce the formation of beta-amyloid 42 peptide, said method comprising:
administering said molecule to the cell line of claims 7-10;
determining the amount of beta-amyloid 42 peptide formed; and
comparing said amount with the amount of beta-amyloid 42 formed in said cell without administering said molecule.

13. A method of screening a molecule for treating Alzheimer's Disease, said method comprising:
administering the molecule to the transgenic animal of claim 11; and
monitoring the transgenic animal for the effects of said molecule on beta-amyloid deposits in its brain and/or neuronal cell death and/or abnormally-phosphorylated tau protein and/or an increase in the number of glial cells and/or reduced behavioral activity tests.

14. A molecule obtained by a screening method according to either of claims 12 or 13.

15. A process for screening of alternative proteases of Amyloid β and/or γ-secretase-homologues and/or γ-secretase-modulators, said process comprising:
transfecting the polynucleotide sequence of any one of claims 1-5 into a presenilin 1 and presenilin 2 negative cell line;
super-transfecting said cell line with a genetic library, and
monitoring the production of beta amyloid production.
